(19) United States
(12) Patent Application Publication
(10) Pub. No.: US 2018/0353421 A1
(43) Pub. Date: Dec. 13, 2018

(54) CLEAR COAT FORMULATIONS FOR USE OVER NAIL POLISH

(71) Applicant: Elementis Specialties, Inc., East Windsor, NJ (US)

(72) Inventors: Wouter IJDO, Yardley, PA (US); Yanhui CHEN, Princeton, NJ (US); Prashant DESHMUKH, Plainsboro, NJ (US); Rajni GUPTA, Princeton, NJ (US); James A. HECK, Robbinsville, NJ (US); Wayne HOYTE, Parlin, NJ (US); Maurice GRAY, Saint Albans, NY (US)

(73) Assignee: Elementis Specialties, Inc., East Windsor, NJ (US)

(21) Appl. No.: 16/007,239

(22) Filed: Jun. 13, 2018

Related U.S. Application Data
(60) Provisional application No. 62/518,728, filed on Jun. 13, 2017.

(51) Int. Cl. A61K 8/85; C08G 63/16; C08J 3/24; A61K 8/37; A61K 8/41; A61Q 3/02; C09D 167/02

(52) U.S. Cl. CPC A61K 8/85; C08G 63/16; C08J 3/24; C08J 2367/02; A61K 8/416; A61Q 3/02; C09D 167/02; A61K 8/375

(57) ABSTRACT

A clear coating composition for use over nail polish. The clear coating composition comprises (a) a crosslinkable coating composition comprising: ingredient A that has at least two protons that can be activated to form a Michael carbanion donor; ingredient B that functions as a Michael acceptor having at least two ethylenically unsaturated functionalities each activated by an electron-withdrawing group; and a dormant carbamate initiator of Formula (1)

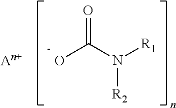

wherein R₁ and R₂ can be independently selected from hydrogen, a linear or branched substituted or unsubstituted alkyl group having 1 to 22 carbon atoms; 1 to 8 carbon atoms; 1 to 3 carbon atoms; and Aⁿ⁺ is a cationic species or polymer and n is an integer equal or greater than 1 with the proviso that Aⁿ⁺ is not an acidic hydrogen; an ingredient D having one or more reactive protons that are more acidic than the two protons of ingredient A, with respect to pKa; at least one organic solvent; with the proviso that the clear coating composition is free of FD&C or D&C dyes, lakes or pigments. The clear coating composition optionally further comprising ammonium carbamate (H₂NR₁R₂⁺⁻OC═ONR₁R₂).

### CROSS-REFERENCE TO RELATED APPLICATIONS

This application claims priority benefit from U.S. Provisional Patent Application 62/518,728 filed Jun. 13, 2017 which is incorporated by reference herein in its entirety.

### FIELD OF THE INVENTION

The invention provides for a clear coat composition, containing a dormant carbamate initiator, for use over nail polish.

### BACKGROUND

The coatings industry continues to develop new chemistries as performance requirements for decorative and functional coatings evolve. Drivers for change are varied and these can include: regulatory controls to reduce VOC emissions, concerns about toxic hazards of coating raw materials, a desire for cost reduction, commitments to sustainability, and a need for increased product effectiveness.

UV nail gel coatings have gained rapid popularity with fashion conscious individuals who apply nail polish to fingernails or toenails to decorate and protect nail plates. UV nail gels can produce coatings that exhibit phenomenal chip resistance and durability when properly applied and cured in comparison to those nail coatings derived from traditional solvent based nail lacquers. The performance difference particularly becomes apparent when the coating is applied on human finger nails and tested for durability. UV nail gel coatings can easily last for two weeks or more and still look like new whereas conventional nail polishes are easily scratched and will chip or peel from the natural nail in one to five days. UV nail gels are typically based on acrylates that cure quickly into dense, crosslinked thermoset coatings within half a minute or so. This is an advantage as the coating becomes almost immediately resistant to denting and scratching. Conventional nail lacquers show significant sensitivity to denting while the solvent evaporates from the coating and this requires great care by the individual as the coating dries and hardens; a process that can take easily fifteen to twenty minutes. However, conventional nail polish is easily removed with solvent whereas it can take some effort to remove a fully cured UV nail gel from the nail surface. An expensive UV light also is required for UV nail gel application and this has limited the success of UV nail gels in the mass market for home use. The expense of a UV light is less of an issue for professional salons where a right balance between service rate and a customers' perception of service is more important. As such, there is a need in the consumer market place for durable nail coatings that can cure quickly but do not require procurement of an UV light.

Highly crosslinked, durable coating compositions can be achieved using Michael addition chemistry. The Michael addition reaction involves the nucleophilic addition of a Michael donor, such as a carbanion or another nucleophile to a Michael acceptor, such as an α,β-unsaturated carbonyl. As such, the base catalyzed addition of activated methylene moieties to electron deficient C═C double bonds are known in coatings applications. Representative examples of suitable materials that can provide activated methylene or methine groups are generally disclosed in U.S. Pat. No. 4,871,822, which resins contain a methylene and/or monosubstituted methylene group in the alpha-position to two activating groups such as, for example, carbonyl, cyano, sulfoxide and/or nitro groups. Preferred are resins containing a methylene group in the alpha-position to two carbonyl groups, such as malonate and/or acetoacetate group-containing materials, malonates being most preferred. The α,β-unsaturated carbonyl typically is an acrylate material and representative materials have been disclosed in U.S. Pat. No. 4,602,061. The Michael reaction is fast, can be carried out at ambient temperatures and gives a chemically stable crosslinking bond without forming any reaction by-product.

A typical crosslinkable coating composition comprises a resin ingredient A (Michael donor), a resin ingredient B (Michael acceptor) and a base to start and catalyze the Michael addition reaction. The base catalyst should be strong enough to abstract, i.e. activate a proton from resin ingredient A to form the Michael donor carbanion species. Since the Michael addition cure chemistry can be very fast, the coating formulator is challenged to control the speed of the reaction to achieve an acceptable balance of pot life, open time, tack free time and cure time. Pot life is defined as the amount of time during which the viscosity of a mixed reactive system doubles. Working life or working time informs the user how much time they have to work with a reactive two part system before it reaches such a high state of viscosity, or other condition, that it cannot be properly worked with to produce an acceptable application result. Gel time is the amount of time it takes for a mixed, reactive resin system to gel or become so highly viscous that it has lost fluidity. The open time of a coating is a practical measure of how much time it takes for a drying or curing coating to reach a stage where it can no longer be touched by brush or roller when applying additional coating material without leaving an indication that the drying or curing coating and newly applied coating did not quite flow together. These indications normally take the form of brush or roller marks and sometimes a noticeable difference in sheen levels. The tack free time is the amount of time it takes for a curing or drying coating to be no longer sticky to the touch, i.e. the time for a system to become hard to the touch, with no tackiness. Cure time is the amount of time it takes for a coating system to reach full final properties.

The Michael reaction starts the very moment when coating resin ingredients A and B are mixed together with a suitable base. Since it is a fast reaction, the material in a mixing pot starts to crosslink and the fluid viscosity starts to rise. This limits the pot life, working time and general use as a coating. A dormant initiator that is essentially passive while coating material remains in a mixing vessel but that activates the Michael addition reaction upon film formation allows for longer pot life and working time, yet would show good open time, tack free time and cure time. Hence, the application of dormant initiator technology can provide the formulator with tools to control the speed of the reaction in order to achieve desirable cure characteristics.

U.S. Pat. No. 8,962,725 describes a blocked base catalyst for Michael addition, which is based on substituted carbonate salts. Preferred Michael donor resins are based on malonate and Michael acceptor resins are acrylates. The substituted carbonates can bear substituents, but these should not substantially interfere with the crosslinking reaction between malonate and acrylate. The carbonate salts release carbon dioxide and a strong base upon activation by means of film formation. The base is either hydroxide or alkoxide. Before practical pot life and gel times are achieved with acceptable curing characteristics, the carbonate requires presence of a certain amount of water in the coating formulation for the blocking of the base to become effective. All disclosed blocked carbonate examples utilize methanol and/or water. However, malonate esters are known to be susceptible to base hydrolysis, particularly when water is present. Hence, the water necessary to block the carbonate base can thus degrade malonate oligomers or polymers at the same time, which in turn can lead to altered coatings performance. The hydrolysis product furthermore can result in undesirable destruction of base catalyst by means of formation of malonate salt; a reaction which is cloaked as longer pot life and gel time. Presence of water can also be quite problematic in certain coatings applications. Wood grain raising is a significant problem when water is present in wood coatings; water penetrates into wood, which causes swelling and lifting of fibers and this leaves a rough surface. Water also can cause flash rust, i.e. appearance of rust spots on a metal surface during drying of newly applied paint that contains water. Longer term rust formation in terms of corrosion may also be a problem when dealing with formulations that contain water.

### SUMMARY OF INVENTION

In one embodiment, the present invention provides for a clear coating composition comprising: (a) a crosslinkable coating composition comprising: ingredient A that has at least two protons that can be activated to form a Michael carbanion donor; ingredient B that functions as a Michael acceptor having at least two ethylenically unsaturated functionalities each activated by an electron-withdrawing group; and a dormant carbamate initiator of Formula (1)

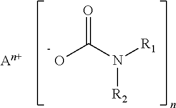

wherein R₁ and R₂ can be independently selected from hydrogen, a linear or branched substituted or unsubstituted alkyl group having 1 to 22 carbon atoms; 1 to 8 carbon atoms; 1 to 3 carbon atoms; and Aⁿ⁺ is a cationic species or polymer and n is an integer equal or greater than 1 with the proviso that Aⁿ⁺ is not an acidic hydrogen; an ingredient D having one or more reactive protons that are more acidic than the two protons of ingredient A, with respect to pKa; at least one organic solvent; optionally further comprising ammonium carbamate (H₂NR₁R₂⁺⁻OC═ONR₁R₂); and with the proviso that the clear coating composition is free of FD&C or D&C dyes, lakes or pigments.

In one embodiment, the present invention provides for a clear coating composition comprising: ingredient A that has at least two protons that can be activated to form a Michael carbanion donor; ingredient B that functions as a Michael acceptor having at least two ethylenically unsaturated functionalities each activated by an electron-withdrawing group; and a carbonate initiator of Formula (3)

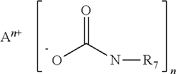

wherein R₇ is selected from hydrogen, a linear or branched substituted or unsubstituted alkyl group having 1 to 22 carbon atoms; 1 to 8 carbon atoms; 1 to 3 carbon atoms; and Aⁿ⁺ is a cationic species or polymer and n is an integer equal or greater than 1 with the proviso that Aⁿ⁺ is not an acidic hydrogen; an ingredient D having one or more reactive protons that are more acidic than the two protons of ingredient A, with respect to pKa; and at least one organic solvent; optionally further comprising ammonium carbamate (H₂NR₁R₂⁺⁻OC═ONR₁R₂). In one embodiment, the clear coat may contain minute quantities of one or more FD&C or D&C dyes, lakes or pigments to counteract unwanted color effects such as yellowing of resin.

In one embodiment, the present invention provides for a method of applying a clear coat over conventional nail lacquer, also referred to as solvent based colored nail polish comprising the step of applying a clear coat composition according to any of claims herein over a one or more layers of solvent based colored nail polish applied to a nail surface, wherein the solvent based colored nail polish comprises solvent, nitrocellulose and at least one of dyes, lakes or pigments.

### DETAILED DESCRIPTION

The invention disclosed here is a clear coat composition comprising a crosslinkable composition comprising a resin ingredient A (Michael donor), a resin ingredient B (Michael acceptor), a dormant initiator ingredient C, ingredient D and organic solvent. In such embodiments, the cross-linkable coating compositions formulated with the dormant initiator can be applied as clear coat over solvent based colored nail polish. For the purposes of this application, solvent based colored nail polish contains solvent, nitrocellulose and at least one of FD&C or D&C dyes, lakes or pigments. Lakes are colorants where one or more of the FD&C or D&C dyes are adsorbed on a substratum, such as alumina, blanc fixe, gloss white, clay, titanium dioxide, zinc oxide, talc, rosin, aluminum benzoate or calcium carbonate.

In practice, consumers may select from their favorite nail colors and apply a conventional nail lacquer as a color coat on nails, and then apply a clear top coat based on the crosslinkable composition as described here, which will provide strength to the coating system as a whole by virtue of the highly crosslinked nature of the cured clear coat. However, this application approach may experience clear coat cure inhibition or the cure may be slowed down significantly to become impractical for use. This can result from an unfavorable interaction between a particular chemical composition of the conventional nail polish and the base catalyzed Michael reaction taking place in a curing clear coat. The tack free time may thus increase, which is undesirable, and clear coat appearance may potentially also suffer. Addition of components slightly more acidic than those encountered in donor resin ingredient A, such as those of ingredient D, can greatly shorten the tack free time to an acceptable time for appeal to consumers. Hence, such additives may in essence actually reduce the coating open time. For example, addition of 1,2,4-triazole to a topcoat formulation comprised of ingredients A, B and C can minimize this cure slow down when a coating of this formulation is applied over a colored nail polish coating.

Resin Ingredient A (Michael Donor):

Resin ingredients A are compounds, oligomers or polymers that contain functional groups that have reactive protons that can be activated to produce a carbanion Michael donor. In one embodiment, the functional group can be a methylene or methine group and resins have been described in U.S. Pat. No. 4,602,061 and U.S. Pat. No. 8,962,725 for example. In one embodiment, resin ingredients A are those derived from malonic acid or malonate esters, i.e. malonate. Oligomeric or polymeric malonate compounds include polyurethanes, polyesters, polyacrylates, epoxy resins, polyamides, polyesteramides or polyvinyl resins each containing malonate groups, either in the main chain or the side chain or in both.

In one embodiment, polyurethanes having malonate groups may be obtained, for instance, by bringing a polyisocyanate into reaction with a hydroxyl group containing ester or polyester of a polyol and malonic acid/malonates, by esterification or transesterification of a hydroxyl functional polyurethane with malonic acid and/or a dialkyl malonate. Examples of polyisocyanates include hexamethylenediisocyanate, trimethylhexamethylene diisocyanate, isophorone diisocyanate, toluene diisocyanate and addition products of a polyol with a diisocyanate, such as that of trimethylolpropane to hexamethylene diisocyanate. In one embodiment, the polyisocyanate is selected from isophorone diisocyanate and trimethyhexamethylene diisocyanate. In another embodiment, the polyisocyanate is isophorone diisocyanate. In some embodiments, hydroxyl functional polyurethanes include the addition products of a polyisocyanate, such as the foregoing polyisocyanates, with di- or polyvalent hydroxyl compounds, including diethyleneglycol, neopentyl glycol, dimethylol cyclohexane, trimethylolpropane, 1,3-propandiol, 1,4-butanediol, 1,6-hexanediol and polyether polyols, polyester polyols or polyacrylate polyols. In some embodiments, the di- or polyvalent hydroxy compounds include diethyleneglycol, 1,3-propanediol, 1,4-butanediol and 1,6-hexanediol. In other embodiments, the di- or polyvalent hydroxyl compounds include diethyleneglycol and 1,6-hexanediol.

In one embodiment, malonic polyesters may be obtained, for instance, by polycondensation of malonic acid, an alkylmalonic acid, such as ethylmalonic acid, a mono- or dialkyl ester of such a carboxylic acid, or the reaction product of a malonic ester and an alkylacrylate or methacrylate, optionally mixed with other di- or polycarboxylic with one or more dihydroxy and/or polyhydroxy compounds, in combination or not with mono hydroxyl compounds and/or carboxyl compounds. In some embodiments, polyhydroxy compounds include compounds containing 2-6 hydroxyl group and 2-20 carbon atoms, such as ethylene glycol, diethyleneglycol, propylene glycol, trimethylol ethane, trimethylolpropane, glycerol, pentaerythritol, 1,4-butanediol, 1,6-hexanediol, cyclohexanedimethanol, 1,12-dodecanediol and sorbitol. In some embodiments, the polyhydroxy compounds include diethylene glycol, propylene glycol, 1,4-butanediol and 1,6-hexanediol. In other embodiments, the polyhydroxy compounds include propylene glycol and 1,6-hexanediol. In certain embodiments, the polyhydroxy may be a primary alcohol and in certain other embodiments, the polyhydroxy may be a secondary alcohol. Examples of polyols with secondary alcohol groups are 2,3-butanediol, 2,4-pentanediol and 2,5-hexanediol and the like.

In one embodiment, malonate group-containing polymers also may be prepared by transesterification of an excess of dialkyl malonate with a hydroxy functional polymer, such as a vinyl alcohol-styrene copolymer. In this way, polymers with malonate groups in the side chains are formed. After the reaction, the excess of dialkyl malonate may optionally be removed under reduced pressure or be used as reactive solvent.

In one embodiment, malonate group or acetoacetate group containing polymers may also be obtained from reaction with malonate or acetoacetonate with polyols, such as those polyols that are commercially sold for reaction with isocyanates to form polyurethane coatings.

In one embodiment, malonic epoxy esters may be prepared by esterifying an epoxy polymer with malonic acid or a malonic monoester, or by transesterifying with a dialkylmalonate, optionally in the presence of one or more other carboxylic acids or derivatives thereof.

In one embodiment, polyamides having malonate groups may be obtained in the same manner as polyesters, at least part of the hydroxyl compound(s) being replaced with a mono- or polyvalent primary and/or secondary amine, such as cyclohexylamine, ethylene diamine, isophorone diamine, hexamethylene diamine, or diethylene triamine.

In some embodiments, such polyamide compounds can be obtained when 12-hydroxystearic acid is reacted with a diamine such as ethylenediamine. Such polyamides have secondary alcohol groups, which can be esterified with malonic acid or malonate in a second reaction step. In some embodiments, other diamines may also be used in the reaction with 12-hydroxystearic acid, for example: xylylenediamine, butylenediamine, hexamethylenediamine, dodecamethylenediamine, and even dimer amine, which is derived from dimer acid. Polyamines may also be used, but in a right stoichiometric ratio as to avoid gelling of the polyamide in the reactor. Lesquerolic acid may also be used in reactions with polyamines to yield polyamides bearing secondary alcohol groups, which can be used in reactions with malonate to form malonate containing compounds. Reactions that yield malonamides are much less desirable.

In some embodiments, the above mentioned malonate resins may be blended together to achieve optimized coatings properties. Such blends can be mixtures of malonate modified polyurethanes, polyesters, polyacrylates, epoxy resins, polyamides, polyesteramides and the like, but mixtures can also be prepared by blending various malonate modified polyesters together. In some other embodiments, various malonate modified polyurethanes can be mixed together, or various malonate modified polyacrylates, or malonate modified epoxy resins, or various malonate modified polyamides, malonate modified polyesteramides.

In certain embodiments, malonate resins are malonate group containing oligomeric esters, polyesters, polyurethanes, or epoxy esters having 1-100, or 2-20 malonate groups per molecule. In some such embodiments, the malonate resins should have a number average molecular weight in the range of from 250 to 10,000 and an acid number not higher than 5, or not higher than 2. Use may optionally be made of malonate compounds in which the malonic acid structural unit is cyclized by formaldehyde, acetaldehyde, acetone or cyclohexanone. In some embodiments, molecular weight control may be achieved by the use of end capping agents, typically monofunctional alcohol, monocarboxylic acid or esters. In one embodiment, malonate compounds may be end capped with one or more of 1-hexanol, 1-octanol, 1-dodecanol, hexanoic acid or its ester, octanoic acid or its esters, dodecanoic acid or its esters, diethyleneglycol monoethyl ether, trimethylhexanol, and t-butyl acetoacetate, ethyl acetoacetate. In one such embodiment, the malonate is end capped with 1-octanol, diethyleneglycol monoethyl ether, trimethylhexanol, t-butyl acetoacetate and ethyl acetoacetate. In another such embodiment, the malonate is end capped t-butyl acetoacetate, ethyl acetoacetate and combinations thereof.

Monomeric malonates may optionally be used as reactive diluents, but certain performance requirements may necessitate removal of monomeric malonates from resin ingredient A.

In some embodiments, resin ingredients A include oligomeric and/or polymeric acetoacetate group-containing resins. In some embodiments, such acetoacetate group-containing resins are acetoacetic esters as disclosed in U.S. Pat. No. 2,759,913, diacetoacetate resins as disclosed in U.S. Pat. No. 4,217,396 and acetoacetate group-containing oligomeric and polymeric resins as disclosed in U.S. Pat. No. 4,408,018. In some embodiments, acetoacetate group-containing oligomeric and polymeric resins can be obtained, for example, from polyalcohols and/or hydroxyl functional polyether, polyester, polyacrylate, vinyl and epoxy oligomers and polymers by reaction with diketene or transesterication with an alkyl acetoacetate. Such resins may also be obtained by copolymerization of an acetoacetate functional (meth)acrylic monomer with other vinyl- and/or acrylic-functional monomers. In certain other embodiments, the acetoacetate group-containing resins for use with the present invention are the acetoacetate group-containing oligomers and polymers containing at least 1, or 2-10, acetoacetate groups. In some such embodiments, such acetoacetate group containing resins should have Mn in the range of from about 100 to about 5000 g/mol, and an acid number of about 2 or less. Resins containing both malonate and acetoacetate groups in the same molecule may also be used.

In another embodiment, the above mentioned malonate group containing resins and acetoacetate group-containing resins may also be blended to optimize coatings properties as desired, often determined by the intended end application.

Structural changes at the acidic site of malonate or acetoacetate can alter the acidity of these materials and derivatives thereof. For instance, pKa measurements in DMSO show that diethyl methylmalonate (MeCH(CO₂Et)₂) has a pKa of 18.7 and diethyl ethylmalonate (EtCH(CO₂Et)₂) has a pKa of 19.1 whereas diethyl malonate (CH₂(CO₂Et)₂) has a pKa of 16.4. Resin ingredient A may contain such substituted moieties and therewith show changes in gel time, open time, cure time and the like. For example, resin ingredient A may be a polyester derived from a polyol, diethyl malonate and diethyl ethylmalonate.

Resin Ingredient B (Michael Acceptor):

Resin ingredients B (Michael acceptor) generally can be materials with ethylenically unsaturated moieties in which the carbon-carbon double bond is activated by an electron-withdrawing group, e.g. a carbonyl group in the alpha-position. In some embodiments, resin ingredients B are described in: U.S. Pat. No. 2,759,913, U.S. Pat. No. 4,871,822, U.S. Pat. No. 4,602,061, U.S. Pat. No. 4,408,018, U.S. Pat. No. 4,217,396 and U.S. Pat. No. 8,962,725. In certain embodiments, resin ingredients B include acrylates, fumarates and maleates. In other certain embodiments, resin ingredient B is an unsaturated acryloyl functional resin.

In some embodiments, resin ingredients B are the acrylic esters of chemicals containing 2-6 hydroxyl groups and 2-20 carbon atoms. These esters may optionally contain hydroxyl groups. In some such embodiments, examples of such acrylic esters include hexanediol diacrylate, trimethylolpropane triacrylate, pentaerythritol triacrylate, di-trimethylolpropane tetraacrylate. In one such embodiment, acrylic esters include trimethylolpropane triacrylate, di-trimethylolproane tetraacrylate, dipentaerythritol hexaacrylate, pentaerythritol ethoxylated (EO)ₙ tetraacrylate, trimethylolpropane ethoxylated (EO)ₙ triacrylate and combinations thereof. In another embodiment, acrylamides may be used as a resin ingredient B.

In other embodiments, resin ingredients B are polyesters based upon maleic, fumaric and/or itaconic acid (and maleic and itaconic anhydride), and chemicals with di- or polyvalent hydroxyl groups, optionally including materials with a monovalent hydroxyl and/or carboxyl functionality.

In other embodiments, resin ingredients B are resins such as polyesters, polyurethanes, polyethers and/or alkyd resins containing pendant activated unsaturated groups. These include, for example, urethane acrylates obtained by reaction of a polyisocyanate with an hydroxyl group-containing acrylic ester, e.g., an hydroxyalkyl ester of acrylic acid or a resins prepared by esterification of a polyhydroxy material with acrylic acid; polyether acrylates obtained by esterification of an hydroxyl group-containing polyether with acrylic acid; polyfunctional acrylates obtained by reaction of an hydroxyalkyl acrylate with a polycarboxylic acid and/or a polyamino resin; polyacrylates obtained by reaction of acrylic acid with an epoxy resin; and polyalkylmaleates obtained by reaction of a monoalkylmaleate ester with an epoxy polymer and/or an hydroxyl functional oligomer or polymer. In certain embodiments, polyurethane acrylate resins may be prepared by reaction of hydroxyalkyl acrylate with polyisocyanate. Such polyurethane acrylate resins independently include bis(2-hydroxyl ethyl acrylate) trimethylhexyl dicarbamate [2-hydroxyethyl acrylate trimethylhexamethylene diisocyanate (TMDI) adduct], bis(2-hydroxyl ethyl acrylate) 1,3,3-trimethylcyclohexyl dicarbamate [2-hydroxyethyl acrylate 1,3,3-trimethylcyclohexyl diisocyanate/isophorone diisocyanate (IPDI) adduct], bis(2-hydroxyl ethyl acrylate) hexyl dicarbamate [2-hydroxyethyl acrylate hexamethylene diisocyanate (HDI) adduct], bis(2-hydroxylethyl acrylate) methylene dicyclohexyl dicarbamate [2-hydroxyethyl acrylate methylene dicyclohexyl diisocyanate (HMDI) adduct], bis(2-hydroxylethyl acrylate) methylenediphenyl dicarbamate [2-hydroxyethyl acrylate methylenediphenyl diisocyanate (MDI) adduct], bis(4-hydroxybutyl acrylate) 1,3,3-trimethylcyclohexyl dicarbamate [4-hydroxybutyl acrylate IPDI adduct], bis(4-hydroxybutyl acrylate) trimethylhexyl dicarbamate [4-hydroxybutyl acrylate TMDI adduct], bis(4-hydroxybutyl acrylate) hexyl dicarbamate [4-hydroxybutyl acrylate HDI adduct], bis(4-hydroxybutyl acrylate) methylene dicyclohexyl dicarbamate [4-hydroxybutyl acrylate HMDI adduct], bis(4-hydroxybutyl acrylate) methylenediphenyl dicarbamate [4-hydroxybutyl acrylate MDI adduct].

In other embodiments, resin ingredients B have unsaturated acryloyl functional groups.

In certain embodiments, the acid value of the activated unsaturated group-containing material (resin ingredient B) is sufficiently low to not substantially impair the Michael addition reaction, for example less than about 2, and further for example less than 1 mg KOH/g.

As exemplified by the previously incorporated references, these and other activated unsaturated group containing resins, and their methods of production, are generally known to those skilled in the art, and need no further explanation here. In certain embodiments, the number of reactive unsaturated group ranges from 2 to 20, the equivalent molecular weight (EQW: average molecular weight per reactive functional group) ranges from 100 to 2000, and the number average molecular weight Mn ranges from 100 to 5000.

In one embodiment, the reactive part of resin ingredients A and B can also be combined in one A-B type molecule. In this embodiment of the crosslinkable composition both the methylene and/or methine features as well as the α,β-unsaturated carbonyl are present in the same molecule, be it a monomer, oligomer or polymer. Mixtures of such A-B type molecules with ingredient A and B are also useful.

Each of the foregoing embodiments of resin ingredient A and resin ingredient B may be combined with the various embodiments of a dormant initiator ingredient C, described below, to arrive at the inventions described herein. In one embodiment, resin ingredient A is a polyester malonate composition and resin ingredient B is a polyester acrylate. In another embodiment, resin ingredient A is a polyurethane malonate composition and resin ingredient B is a polyester acrylate. In another embodiment, resin ingredient A is a polyurethane malonate composition and resin ingredient B is a polyester acrylate. In another embodiment, resin ingredient A is a polyurethane malonate composition and resin ingredient B is a polyurethane acrylate. In another embodiment, resin ingredient A is a polyester malonate having acetoacetate end groups and resin ingredient B is a polyester acrylate. In yet another embodiment, resin ingredient A is a polyester malonate having acetoacetate end groups and resin ingredient B is a polyurethane acrylate. In still yet another embodiment, resin ingredient A is a polyester malonate having acetoacetate end groups and resin ingredient B is a mixture of polyester acrylate and polyurethane acrylate.

In the foregoing embodiments, the number of reactive protons for resin ingredients A, and the number of α,β-unsaturated carbonyl moieties on resin ingredient B can be utilized to express desirable ratios and ranges for resin ingredients A and B. Typically, the mole ratio of reactive protons of ingredient A that can be activated with subsequent carbanion formation relative to the activated unsaturated groups on ingredient B is in the range between 10/1 and 0.1/1, or between 4/1 and 0.25/1, or between 3.3/1 and 0.67/1. However, the optimal amount strongly depends also on the number of reactive groups present on ingredients A and/or B.

The amount of dormant initiator used, expressed as mole ratio of protons that can be abstracted to form an activated Michael donor species (e.g. the methylene group of malonate can provide two protons for reactions, while a methine group can provide one proton to form an activated species) relative to initiator, ranges from about 1000/1 to 1/1, or from 250/1 to 10/1, or from 125/1 to 20/1 but the optimal amount to be used depends also on the amount of solvent present, reactivity of various acidic protons present on resin ingredients A and/or B.

Dormant Initiator Ingredient C:

In one embodiment, the dormant initiator is a carbamate salt with a structure shown in Formula 1:

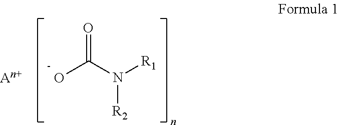

R₁ and R₂ can be independently selected and is hydrogen or an alkyl group with 1 to 22 carbon atoms. The alkyl group can be linear or branched. Shorter alkyl chains with 1 to 8 carbon atoms are preferred. Most preferred is a methyl group, but ethyl, propyl, butyl groups also are preferred. Unsubstituted alkyl chains are preferred, but hydroxyl groups are preferred when one or both alkyl chains are substituted. Aⁿ⁺ is discussed below. Dormant carbamate initiator ingredient C is significantly slow in promoting the Michael reaction prior to applying the crosslinkable composition of this invention as a coating so it can be regarded as essentially inactive, or minimally active, yet the initiator initiates Michael addition once the coating is applied as a film.

The carbamate initiator releases carbon dioxide and ammonia or an amine upon activating resin ingredient A by means of a shift in equilibrium. The overall activation equilibrium reaction is illustrated in equation 1 for example with a malonate material (R′ and R″ can be the same or different and can be an alkyl or a malonate containing polymer). The activation process produces the carbanion Michael donor.

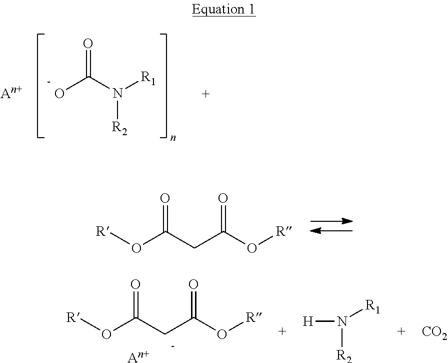

The carbanion can react with the Michael acceptor, an acrylate for example, to yield a malonate-acrylate adduct, which is very basic and is readily protonated, typically by another malonate methylene or methine moiety thus restarting another cycle and continuing the Michael addition process. Solvent potentially can participate in the Michael addition cycle. The equilibrium of equation 1 can be shifted according to Le Châtelier's principle when ammonia or amine and carbon dioxide are allowed to leave the system therewith driving the Michael addition reaction. However, the carbon dioxide and the ammonia or amine that are formed in equation 1 react exothermally with each other at a fast rate to form an ammonium carbamate in an equilibrium reaction that favors formation of the ammonium carbamate. This equilibrium reaction is shown in equation 2.

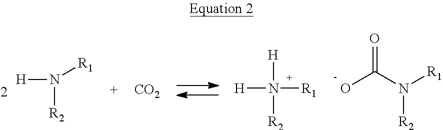

The protonated ammonium cation is a more acidic species (pKₐ≈9) than the malonate methylene group (pKₐ≈13) and preferentially reacts with a carbanion such as the malonate-acrylate adduct or the Michael donor carbanion of ingredient A for example. The initial carbamate initiator salt reforms in this reaction step. This process is illustrated in equation 3.

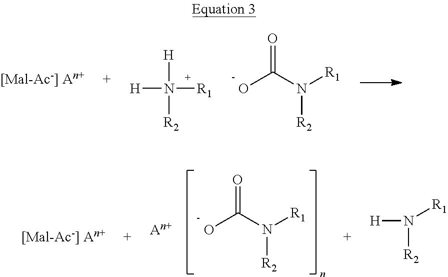

The carbamate initiator thus is able to start the Michael addition cycle by means of a shift in equilibrium, but its decomposition products push back on the equilibrium and can react and stop the Michael reaction and regenerate the carbamate initiator as long as amine and carbon dioxide are available. This ensures long pot life and gel time of the coating composition. Once the coating composition is applied on a substrate, the amine and carbon dioxide can escape into the atmosphere above the coating film and therewith unleash the full speed potential of the Michael addition reaction.

Only ammonia, primary and secondary amines can react with carbon dioxide to form ammonium carbamate material. Tertiary amines do not react with carbon dioxide to form carbamates. However, ammonia, and amines can also react with acrylates at ambient conditions albeit at different rates and these competing aza-Michael additions are illustrated in equation 4.

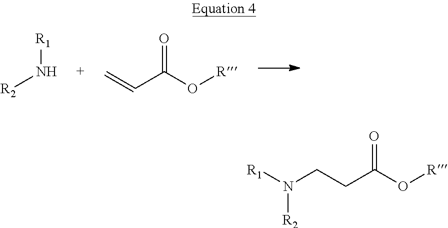

The inventors surprisingly found the carbamate initiator of formula 1 to be dormant in the crosslinkable composition of this invention despite the reaction shown in equation 4, which has the potential to drive a shift in equilibria. The reactions shown in equation 1, 2, 3 and 4 can be utilized to fine tune overall pot life, open time, cure rate and gel time. The reaction shown in equation 4 has an advantage in that it can remove undesirable amine odor from the curing coating as the dormant carbamate initiator activates.

Additional amine functional groups can optionally be added to the coating formulation to impact pot life, open time, cure rate and gel time. In another approach, both a quaternary ammonium carbamate, (A^{+-OC═ONR}₁R₂), as well as an ammonium carbamate, (H₂NR₁R₂⁺⁻OC═ONR₁R₂), may be used together as a dormant initiator system. In yet another approach, excess carbon dioxide may be utilized to influence equilibria according to Le Châtelier's principle and thus influence pot life, open time, cure time and the like. Another surprising result of this invention involves the dormant carbamate initiator and its interaction with acetoacetylated resins. Dormancy is preserved despite the fact that amines rapidly react with acetoacetic esters to yield a resin with enamine functionalities. Enamine and ketamines are tautomers. The two isomers readily interconvert with each other, with the equilibrium shifting depending on the polarity of the solvent/environment. The enamine and ketamine groups convey increased methine/methylene acidity and the resin can crosslink in a reaction with α,β-unsaturated resins via Michael addition but the reactivity depends on the enamine/ketamine equilibrium. However, once the dormant carbamate initiator actives upon film formation and releases amine and carbon dioxide, the amine may preferentially react with acrylate or acetoacetate moieties in competing reactions, and thus significantly alter the crosslinking reaction characteristics during these initial stages when amine becomes available. The coating formulator thus has additional tools available by making use of the rich reaction chemistry that the amine offers by, for instance, using a mix of acetoacetate and malonate functional groups.

In another embodiment of the invention, polyamines, potentially in combination with monoamines, may also be utilized as raw material for ammonium carbamate salt formation. In such embodiments, dormant carbamate initiator systems may also be derived from such carbamates when at least a part of the protonated ammonium cations in these ammonium carbamate salts are replaced for quaternary ammonium cations, or other cationic species, or cationic polymers using synthetic approaches described above. For instance, piperazine is known to have a high capacity for carbon dioxide capture and shows a high heat of absorption as well. Piperazine forms various carbamates, e.g. protonated piperazine carbamate, piperazine carbamate and/or piperazine bicarbamate salts with mono or di protonated piperazine. The formation/decomposition equilibrium of carbamates is temperature dependent and varies depending on the amine employed as well as solvent/environment. In another embodiment, carbamates may be derived from pyrrolidine, 2-methylpyrrolidine, 3-methylpyrrolidine, piperidine, piperazine, methylethanolamine, diethanolamine, isopropanolamine, diisopropanolamine.

In yet another embodiment, carbamates may be derived from amines that have a pKa greater than 7, or carbamates derived from amines that have a pKa greater than 8, or carbamates derived from amines that have a pKa greater than 9, or carbamates that are derived from amines that have a pKa greater than 10.

In another embodiment, the dormant initiator has a structure as shown in Formula 2:

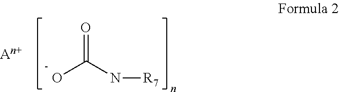

Wherein R₇ can be independently selected and is hydrogen or an alkyl group with 1 to 22 carbon atoms. The alkyl group can be linear or branched. Shorter alkyl chains with 1 to 8 carbon atoms are preferred. Most preferred is an ethyl group, but methyl, propyl, butyl groups also are preferred. Unsubstituted alkyl chains are preferred, but hydroxyl groups are preferred when an alkyl chain is substituted.

Aⁿ⁺ is a cationic material and n is an integer equal or greater than 1. Aⁿ⁺ can be a monovalent cation, such as an alkali metal, earth alkali metal or another monovalent metal cation, a quaternary ammonium or a phosphonium compound. Aⁿ⁺ can also be a multivalent metal cation, or a compound bearing more than one quaternary ammonium or phosphonium groups, or can be a cationic polymer. Aⁿ⁺ preferably is a monovalent quaternary ammonium compound where n is 1. Aⁿ⁺ is not an acidic hydrogen.

In certain embodiments, Aⁿ⁺ of formulas 1 and 2 is a monovalent quaternary ammonium compound and the structure of this cation is shown in formula 3. A large selection of such quaternary ammonium compounds is commercially available from various manufacturers. In some embodiments, quaternary ammonium compounds may be derived from tertiary amines and in certain embodiments, quaternized with a methyl or benzyl group. In other embodiments, tetra alkyl ammonium compounds also can be used. R₃, R₄ and R₅ are independently selected and are linear or branched alkyl chains having from 1 to 22 carbon atoms. In some such embodiments, ammonium compounds where R₃, R₄ and R₅ are independently selected and range from 1 to 8. In some other such embodiments, ammonium compounds can be identified within this group and is dependent upon performance and raw materials costs. In certain embodiments, R₆ is a methyl or a benzyl group or an alkyl group having from 1 to 22 carbon atoms or 2 to 6 carbon atoms. The quaternary ammonium compound is commercially available as a salt and the anion typically is chloride, bromide, methyl sulfate, or hydroxide. Quaternary ammonium compounds with methylcarbonate or ethylcarbonate anions are also available.

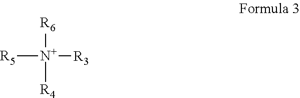

Examples of Aⁿ⁺ of formulas 1 and 2 include dimethyldiethylammonium, dimethyldipropylammonium, triethylmethylammonium, tripropylmethylammonium, tributylmethylammonium, tripentylmethyl ammonium, trihexylmethylammonium tetraethylammonium, tetrapropylammonium, tetrabutyl ammonium, tetrapentylammonium, tetrahexylammonium, benzyltrimethyl ammonium, benzyltriethylammonium, benzyltripropylammonium, benzyltributyl ammonium, benzyltripentyammonium, and benzyltrihexylammonium.

The crosslinkable composition of this invention preferably contains some solvent. The coating formulator may choose to use an alcohol, or a combination of alcohols as solvent for a variety of reasons. This is not a problem for the carbamate initiator, and regeneration thereof, because ammonia as well as primary and secondary amines react much faster with carbon dioxide than hydroxides or alkoxy anions. Other solvents like ethylacetate or butylacetate may also be used, potentially in combination with alcohol solvents. Ethanol is a preferred solvent. Isopropyl alcohol also is a preferred solvent. Methanol is not preferred as a solvent because of health and safety risks, and is particularly not preferred and cannot be used when the crosslinkable composition is used as a coating for finger nails and toe nails. Other oxygenated, polar solvents such as ester or ketones for instance, are also suitable and can be used, potentially in combination with alcohol. Other organic solvents may also be used. Although water is not preferred, water may be present in the solvent, either deliberately added, or produced in situ in minor quantities during preparation of the dormant initiator. The crosslinkable composition of this invention may also be formulated without solvent in some cases. The crosslinkable coating contains typically at least 5 wt % of solvent, preferably between 5% and 45%, more preferable between 5% and 35%, but preferable not more than 60% because of VOC restrictions.

The dormant carbamate initiator salt may be prepared in various ways. One approach is by means of ion exchange. In this approach, a cation exchange column is charged with quaternary ammonium ions, which in turn can replace the protonated amine of an ammonium carbamate so that a quaternary ammonium carbamate solution is obtained. For instance, a concentrated solution of tributylmethylammonium chloride in water is passed through a cation exchange column. Next, the column is washed free of excess salt and rinsed with anhydrous alcohol to remove any residual water. In a next step, dimethylammonium dimethyl carbamate, NH₂(CH₃)₂⁺⁻C═ON(CH₃)₂, optionally diluted with alcohol, is passed through the column so as to obtain a tributylmethylammonium dimethyl carbamate solution in alcohol. A similar approach with anionic ion exchange columns may be devised. The solution can be titrated with base or acid to assess the initiator concentration and whether the dormant initiator formation has been successful. Such analytical reactions are well known to one skilled in the art and need not be further described here.

In a most preferred approach, an ammonium carbamate solution may be treated with a strong base in alcohol. For example, dimethylammonium dimethyl carbamate is mixed with one molar equivalent of a tetrabutylammonium hydroxide dissolved in ethanol. This yields a tetrabutylammonium dimethyl carbamate solution after the neutralization reaction, as well as dimethyl amine and water. An excess of dimethylammonium dimethyl carbamate may also preferably be used to ensure no residual hydroxide is left in the initiator solution and/or to increase pot life and gel time.

In another approach, dimethylammonium dimethyl carbamate, is treated with an alcoholic solution of potassium t-butoxide to yield a solution of potassium dimethyl carbamate, dimethylamine and t-butanol.

In yet another preferred approach, a diethylmalonate solution in ethanol is treated with a quaternary ammonium ethoxide prior to adding dimethylammonium dimethyl carbamate to yield a quaternary ammonium dimethyl carbamate solution in ethanol mixed with diethylmalonate and dimethylamine. In yet another approach, a quaternary ammonium hydroxide base, such as for instance, tetrabutylammonium hydroxide is added to a solution of diethylmalonate in ethanol. Next, dimethylammonium dimethyl carbamate is added to yield a tetrabutylammonium dimethyl carbamate solution mixed with diethylmalonate, dimethylamine and water. In yet another approach, a strong alkoxide base like sodium ethoxide is added to a solution of diethylmalonate in ethanol. Next, a quaternary ammonium chloride salt is added, for instance tributylmethylammonium chloride, and the solution is filtered to remove sodium chloride salt. Next, a stoichiometric amount of dimethylammonium dimethylcarbamate is added to yield a solution of diethylmalonate, tributylmethylammonium carbamate and dimethylamine in ethanol. Malonate resin ingredient A may also be used in such reactions. In a very preferred approach, optionally in the presence of an organic solvent, resin ingredient A is first treated with a quaternary ammonium base, preferably a quaternary ammonium hydroxide solution, before adding an ammonium carbamate, potentially in excess, to yield a mixture of resin ingredient A, quaternary ammonium carbamate and amine.

Other dialkyl ammonium dialkyl carbamates, or monoalkyl ammonium monoalkyl carbamates or ammonium carbamate or mixtures thereof may also be used but those derived from smaller amines are preferred. Ammonium carbamates are readily prepared by reacting carbon dioxide with ammonia or amine. Mixtures of amines can also be used to prepare ammonium carbamate(s). Carbamate metal salt solutions can also be prepared as described in U.S. Pat. No. 5,808,013.

The dormant carbonate initiator salt may be prepared in various ways, for instance by reacting diethyl carbonate, quaternary ammonium hydroxide or quaternary ammonium alkoxide in ethanol in the presence of some water. In another approach, the carbonate initiator salt is obtained by reacting carbon dioxide with a solution of quaternary ammonium hydroxide or quaternary ammonium alkoxide in ethanol. The alkoxide is a conjugate base of an alcohol and examples of the alkoxide include ethoxide, isopropoxide and tert-butoxide.

### Ingredient D

In one embodiment, the crosslinkable composition of this invention comprising ingredients A, B and C may optionally contain an additional ingredient D, which once activated, can react with the Michael acceptor. In one such embodiment, ingredient D has one or more reactive protons that are more reactive, i.e. more acidic than those of ingredient A (the pKa of ingredient D is lower than that of ingredient A) yet not as reactive as ammonium carbamate with respect the pKa. In another embodiment, ingredient D may be more acidic than ammonium carbamate with respect to pKa. In such embodiments, the reactive protons of ingredient D are present at a fraction based on the reactive protons of ingredient A where the fraction ranges from 0 to 0.5, or from 0 to 0.35, or between 0 and 0.15.

Examples of ingredient D include; succinimide, isatine, ethosuximide, phthalimide, 4-nitro-2-methylimidazole, 5,5-dimethylhydantioin, phenol, 1,2,4-triazole, ethylacetoacetate, 1,2,3-triazole, ethyl cyanoacetate, benzotriazole, acetylacetone, benzenesulfonamide, 1,3-cyclohexanedione, nitromethane, nitroethane, 2-nitropropane, diethylmalonate, 1,2,3-triazole-4,5-dicarboxylic acid ethyl ester, 1,2,4-triazole-3-carboxylic acid ethyl ester, 3-Amino-1,2,4-triazole, 1H-1,2,3-triazole-5-carboxylic acid ethyl ester, 1H-[1,2,3]triazole-4-carbaldehyde, morpholine, purines such as purine, adenine, guanine, hypoxanthine, xanthine, theobromine, caffeine, uric acid and isoguanine; pyrimidines, such as thymine and cytosine; uracil, glycine, ethanimidamide, cysteamine, allantoin, N,N-dimethylglycine, allopurinol, N-methylpyrrolidine, benzeneboronic acid, salicyl aldehyde, 3-hydroxybenzaldehyde, 1-naphthol, methylphenidate and Vitamin E.

In another embodiments, ingredient D may be incorporated into resin ingredient A. In such embodiments, substituted succinimides, including hydroxyl group containing succinimide derivatives, 3-hydroxy-2,5-pyrrolidinedione and 3-(hydroxymethyl)-2,5-pyrrolidinedione, or carboxylic acid group containing succinimide derivative, 2,5-dioxo-3-pyrrolidinecarboxylic acid can undergo condensation reactions with either acid/ester groups or hydroxyl groups at the end of resin A polymer chain, where the succinimide moiety will be incorporated into the polymer backbone as end cap.

### Formulation of Crosslinkable Composition

The crosslinkable composition useful as a coating can be formulated as a one component, a two component system or a three component system. In an embodiment of a two component system, initiator ingredient C is added to a mixture of ingredients A and B just prior to use; ingredient D may optionally be added to the initiator ingredient C or the mixture of ingredients A and B. In an alternative embodiment, ingredients A and C are mixed, and ingredient B is added prior to use ingredient; D may optionally be added to the mixture of ingredient A and initiator ingredient C or ingredient B. In yet another embodiment, ingredient A is added to a mixture of ingredients B and C prior to use; ingredient D may optionally be added to ingredient A or the mixture of ingredient B and initiator C. In certain embodiments, pot life, working time and gel time can be adjusted by selection of the initiator structure, the amount used in the crosslinkable composition, presence of additional ammonium carbamate and to a certain extent the amount of solvent and/or water. A gel time of hours, and even days can be readily achieved, and gel times of weeks are possible. As such, the dormant initiator allows for an opportunity to formulate a three component paint system. In such embodiment of a one component system, ingredients A, B, C and D are mixed together, optionally with other ingredients to formulate a paint, which is then canned and stored until use. In certain embodiments, a one component system can be enhanced by means of using excess carbon dioxide gas over the crosslinkable composition as to further improve pot life and gel time. For instance, a paint composition formulated according to the invention may have a protective atmosphere of carbon dioxide over the paint volume; and in yet another embodiment, a container containing the crosslinkable composition may even be pressurized with carbon dioxide. In another embodiment, a one component system containing ingredients A, B and C are in a container filled to capacity with essentially no space remaining for other liquid or gaseous ingredients. In yet another embodiment, additional ammonium carbamate may further enhance stability in such one component coating formulations.

In another embodiment, the present invention provides for the crosslinkable coating composition wherein ingredient A, ingredient B and the carbamate initiator are contained in a container having two or more chambers, which are separated from one another. In one such embodiment, ingredient A and ingredient B are contained in separate chambers to inhibit any reaction. In another such embodiment, the carbamate initiator is contained in the chamber having ingredient A, and optionally containing CO₂ and/or ammonium carbamate. In another such embodiment, the carbamate initiator is contained in the chamber having ingredient B, and optionally containing CO₂ and/or ammonium carbamate.

In another embodiment, the present invention provides for the crosslinkable coating composition such that ingredient A and ingredient B are contained in the same chamber and the carbamate initiator is contained in a separate chamber to inhibit any reaction and said separate chamber optionally containing CO₂ and/or ammonium carbamate.

In another embodiment, the present invention provides for the crosslinkable coating composition wherein ingredient A and ingredient B and carbamate initiator are contained in a container having a single chamber, wherein the container optionally contains CO₂ and/or ammonium carbamate. Malonate esters are known to be susceptible to base hydrolysis, particularly when water is present. Water potentially can lead to undesirable destruction of initiator by means of formation of malonate salt and it can degrade malonate oligomers or polymers, which in turn can lead to altered coatings performance. Transesterification reactions also can occur with malonate esters and alcohol solvent. These reactions potentially can be limiting to the formulation of an acceptable working life, as a coatings formulator seeks to increase pot life and gel time for a crosslinkable composition formulated either as a one or two component system. However, primary alcohols such as methanol and ethanol are much more active in transesterification reactions than secondary alcohols such as isopropanol, while tertiary alcohols are generally least active. Furthermore, additional resistance towards hydrolysis and transesterification can be obtained when malonate polyester resins are derived from malonic acid, or a dialkylmalonate such as diethylmalonate, and polyols bearing secondary alcohol groups; such as 2,3-butanediol, 2,4-pentanediol and 2,5-hexanediol and the like. The combination of such polyester resins and non-primary alcohol solvents, such as isopropanol or butanol, is particularly useful in achieving desirable resistance towards transesterification reactions. In a preferred approach, resin ingredient A comprises malonate moieties that have been esterified with polyols bearing secondary alcohol groups and where secondary alcohol is present as solvent in the crosslinkable composition of this invention. In yet another approach, tertiary alcohols are used as solvent or solvents as used that do not participate in transesterification reactions. Other resins may also be formulated using such stabilizing approaches towards resin breakdown and such approaches are well known to one skilled in the art and need not be further described here.

The number of reactive protons for ingredients A, and the number of α,β-unsaturated carbonyl moieties on resin ingredient B can be utilized to express desirable ratio's and ranges for ingredients A and B. Typically, the mole ratio of reactive protons of ingredient A that can be activated with subsequent carbanion formation relative to the activated unsaturated groups on ingredient B is in the range between 10/1 and 0.1/1, preferably between 4/1 and 0.25/1, and more preferably 3.3/1 and 0.67/1. However, the optimal amount strongly depends also on the number of such active functionalities present on ingredients A and/or B. Although good tack free time may be obtained over a wide ratio range, coatings properties, such as hardness for instance may show a smaller preference range.

The crosslinkable composition of this invention comprising ingredients A, B and C may optionally contain an additional ingredient D, which once activated, can react with the Michael acceptor. Ingredient D has one or more reactive protons that are more reactive, i.e. more acidic than those of ingredient A (the pKa of ingredient D is lower than that of ingredient A) yet not as reactive as ammonium carbamate with respect the pKa. In some occasions, ingredient D may be more acidic than ammonium carbamate with respect to pKa. The reactive protons of ingredient D are present at a fraction based on the reactive protons of ingredient A. The fraction ranges from 0 to 0.5, more preferably from 0 to 0.35, even more preferable between 0 and 0.15.

The amount of initiator used, expressed as mole ratio of protons that can be abstracted to form an activated Michael donor species (e.g. the methylene group of malonate can provide two protons for reactions, while a methine group can provide one proton to form an activated species) relative to initiator, ranges from about 1000/1 to 1/1, more preferably from 250/1 to 10/1, even more preferable from 125/1 to 20/1.

Certain embodiments of the formulation may optionally comprise resins, such as, but not limited to nitrocellulose, polyvinylbutyral and/or tosylamide formaldehyde resins. Such resins may act as film formers, adhesion promoters, and aids to removal. These resins may also qualify as solvent-dissolvable resins.

The cross-linkable coating composition of this invention can comprise additives such as wetting agents, defoamers, rheological control agents, ultraviolet (UV) light stabilizers, dispersing agents, flow and leveling agents, optical brighteners, gloss additives, radical inhibitors, radical initiators, adhesions promotors, plasticizers and the like.

The crosslinkable composition of this invention formulated as a clear coat may be packaged in a single unit package good for one time use. Such single serve units contain enough coating material to clear coat all finger and toe nails. A single use package may contain a clear coat formulated as a one component system where all ingredients are mixed in one chamber, potentially with extra ammonium carbamate and carbon dioxide to push back on the dormant carbamate initiator. The single unit package may contain more than one chambers when the clear coat system is formulated as a multi component system, e.g. two chambers when the clear coat is formulated as a two component system, or three chambers when ingredients A, B and C are all kept separate until use. Packages are known where a seal between chambers is broken to allow for materials to be mixed in the merged chambers and a proper ratio of components is maintained by virtue of the design of the package. Flexible packages and more rigid containers such as bottles that have more than one chamber where contents can be mixed upon demand are known and are readily available. Single unit packages may also include a brush for application. In another approach deviating from a single use concept, material may be dispensed from a single chamber (flexible) package that can be resealed. Multi chamber package that utilize plungers are also known and proper mixing of components can be insured by use of a mixing nozzle for instance. Material may be dispensed multiple times provided the time between uses does not exceed the working life of the clear coat in a mixing chamber or if the working life is to be exceeded, the mixing nozzle is removed and the package capped and stored until future use when a new mixing nozzle will be used. Many packaging solutions are available from packaging providers and these are well known to one skilled in the art.

The following examples further describe and demonstrate illustrative embodiments within the scope of the present invention. The examples are given solely for illustration and are not to be construed as limitations of this invention as many variations are possible without departing from the spirit and scope thereof.

### Example 1

### Synthesis of Carbamate Initiator by Means of Ion Exchange.

A glass column fitted with a frit at the bottom was charged with 55 g of Amberlite IR 120 Na cation exchange resin, which was then swollen with distilled water. The resin was then washed 3 times with 200 ml water, and charged with 10 wt. % of tributylmethylammonium chloride (TBMA Cl) in water solution. To maximize the ion exchange, the charging process was repeated three times. The ion exchange efficiency was followed gravimetrically. After charging the resin with tributylmethylammonium (TBMA) cations and washing free of excess TBMA Cl, the resin was made water free by washing it with anhydrous ethanol. Washing was continued until the water content of the wash ethanol fell below 0.07 wt % as determined by coulometric Karl-Fischer titration. Next, a 10 wt. % solution of dimethylammonium dimethylcarbamate (DMA DMC) in anhydrous ethanol was passed through the charged resin. Not more than 35% of the resin ion exchange capacity was utilized to ensure a complete conversion of DMA DMC. The tributylmethylammonium dimethylcarbamate (TBMA DMC) initiator was characterized by nuclear magnetic resonance (NMR) analysis and Fourier transform infrared spectroscopy (FTIR) and was titrated with acid and base to assess concentration. In a similar manner, TBMA DMC carbamate initiators were prepared in 1-propanol and 2-propanol.

### Example 2

### Synthesis of Carbamate Initiator by Neutralization of Malonate Carbanion.

To a 250 ml single neck round-bottom flask was charged 5.0 g of diethyl malonate (DEM) and 28.2 g of a 1.0 M solution of potassium t-butoxide (PtB) in tetrahydrofuran (THF). A white precipitate was immediately observed. At the end of addition, 50.0 g of anhydrous isopropanol was added to the reaction mixture under constant stirring to obtain a homogeneous white suspension. Then 7.36 g of dry TBMA Cl was mixed into the flask, stirring was continued for another 10-15 minutes before 4.19 g of DMA DMC was added. The reaction mixture was continuously stirred at room temperature for one hour, and white suspension was removed by filtration and a clear carbamate initiator solution was obtained free of water.

### Example 3

### General Synthesis of Carbamate Initiator by Neutralization of Quaternary Ammonium Hydroxide.

Most of the methanol solvent from a 40 g tetrabutylammonium hydroxide (TBA OH) solution in methanol (1 M) was removed with a rotary evaporator. The material was not allowed to become completely dry without solvent as dry quaternary ammonium hydroxide base is susceptible to decomposition. Next, 40 grams of ethanol was added and most of the solvent was again removed. This procedure was repeated at least two more times until the methanol effectively had been replaced as determined by NMR. The solution strength was determined by titration (typically 1.7 mmol base/g solution). Solvent exchange was also carried out to prepare TBA OH solutions in methanol (typical concentration 1.2 mmol/g solution), 1-propanol (typical concentration of 1.1 mmol base/g solution) and TBA OH in 2-propanol (typical concentration of 1.3 mmol base/g solution). Next, about 25 g of TBA OH in ethanol was mixed with DMA DMC in a 1.0:1.1 molar ratio respectively at room temperature and stirred for 1 hour using a magnetic stirrer. The TBA DMC solution in ethanol was light yellow and was characterized by means of acid and base titrations (potentiometric and with indicator), back titrations and NMR. In a similar manner, TBA DMC solutions were prepared in methanol, 1-propanol and 2-propanol. These initiators were designated as initiator II and the alkanol name was used to indicate the alcohol solvent. TBA DMC solutions in the four alcohols were also prepared using a 1.0:1.5 molar ratio of TBA OH and DMA DMC respectively, and these initiators were designated as initiator III and again, the alkanol name was used to indicate the alcohol solvent.

### Example 4

### General Synthesis of Carbonate Catalyst.

The methanol solvent of TBA OH solution (1 M) in methanol was replaced with ethanol as described in example 3. Next, a precise amount of the TBA OH in solution was mixed with diethyl carbonate (DEtC) in a 1:5 molar ratio respectively and stirred for 1 hour at room temperature using magnetic stirrer. The final clear catalyst solution was analyzed by means of titration and NMR. In a similar manner, clear solutions were obtained in 1-propanol and 2-propanol. A solution made using the TBA OH base in methanol resulted in white precipitate which was removed by centrifuge followed by filtration using 0.45μ syringe filter. In a similar approach, catalyst solutions were prepared in various alcohols using TBA OH and dimethylcarbonate (DMeC). Transesterification reaction products were observed in the NMR for all cases where the carbonate alkyl group was different from the solvent, e.g. ethanol formation was observed when DEtC was added to TBA OH in isopropanol and isopropyl groups associated with carbonates were also observed.

### Example 5

### Malonate Resin (I) Synthesis.

A 3 liter reactor was charged with 500 g of diethylene glycol and 1509 g of diethyl malonate (DEM). The reactor was equipped with a Dean-Stark apparatus, mechanical stirrer, nitrogen flow and heating equipment. The mixture was heated to about 180° C. with stirring under nitrogen atmosphere. During a four hour reaction time, about 450 ml of ethanol was collected. Next, the temperature was reduced to 115° C. and a vacuum distillation was initiated to remove about 246 g of DEM. The final product was a lightly yellow colored liquid with less than 0.15 wt. % of residual DEM as determined by gas chromatography (GC). Gel permeation chromatography (GPC) analysis showed three peak molecular weight of 900, 600 and 400 g/mole and the malonate methylene equivalent molecular weight of 156 g/mole.

### Example 6

### Malonate Resin (II) Synthesis.

A reactor was charged with 600 g of polyethylene glycol (PEG 300) and 640 g of DEM and the reaction synthesis procedure was followed from example 5. The reaction yielded a total of about 170 ml of ethanol and 118 g of DEM was removed by distillation. Analysis shows that the light yellow product contains less than 0.1 wt. % of DEM, Mn-1000 g/mole and malonate methylene equivalent molecular weight of 292 g/mole.

### Example 7

### Malonate Resin (III) Synthesis.

A reactor was charged with 30 g of trimethylolpropane (TMP), 107 g of DEM and 17.7 g of tert-butyl acetoacetate (tBAA) and the reaction synthesis procedure was followed from example 5. The reaction resulted in about 25 g of alcohol and 36 g of material was removed by distillation. The light yellow product contained <0.1% of DEM, Mn-2100 g/mole and malonate methylene equivalent molecular weight of 142 g/mole.

### Example 8

### Malonate Resin (IV) Synthesis.

A reactor was charged with 40 g of glycerol (Gly), 68.71 g of DEM and 69.5 g of tBAA were charged to the reactor and the reaction synthesis procedure was followed from example 5. The reaction resulted in 45 g of alcohol collection and 3 g of material was removed by distillation. The light yellow product contained <0.1% of DEM, Mn-1400 g/mole and malonate methylene equivalent molecular weight of 145 g/mole.

### Example 9

### Acetoacetate Modified Polyol.

A reactor (500 ml capacity) was charged with 175 g of STEPANPOL® PC-2011-225 (a commercial polyol resin with hydroxyl value of 225 mg of KOH/g of sample), and 133 g of tertiary butyl acetoacetate. The reactor was equipped with Dean-Stark apparatus, mechanical stirrer, nitrogen flow and heating equipment. The mixture was heated to about 180° C. with stirring under nitrogen atmosphere. In four hours, 55 ml of alcohol was collected and no further distillate was coming out. The reaction temperature was lowered to 115° C. and a vacuum distillation resulted in collection of a total 6 g of tertiary butyl acetoacetate. The final product was light yellow colored with methylene equivalent molecular weight of 306 g/mole (calculated based on the theoretical mole ratio and the tertiary butanol and tertiary butyl acetoacetate collected amount).

### Example 10

### General Synthesis of Carbamate Initiator by Neutralization of Quaternary Ammonium Ethoxide.

Tributylmethylammonium chloride (TBMA Cl), 10 g, was dissolved in ethanol and mixed in 1:1 molar ratio with a 20 wt. % solution of potassium ethoxide in ethanol. The mixture was allowed to stir for 30 min, and the precipitate was then removed by centrifugation. The concentration of TBMA ethoxide thus obtained was determined potentiometrically by means of titration with 0.1 N HCl solution. The typical concentration of TBMA ethoxide thus obtained was about 1.1 mmol/g. Next, about 25 g of TBMA ethoxide in ethanol was mixed with DMA DMC in a 1.0:1.1 molar ratio respectively at room temperature and stirred for 1 hour using a magnetic stirrer. The TBMA DMC solution in ethanol was light yellow in color and was characterized by means of acid and base titrations (potentiometric and with indicator) and NMR.

### Coating Testing

Tack free time was evaluated by lightly pressing a gloved index finger periodically onto the coating. The time when visible marks in the film are no longer left by the pressed finger, is then recorded as the tack free time.

Gel time is taken as the amount of time it takes for a mixed, reactive resin system to gel or become so highly viscous that it has lost fluidity. Typically, the various ingredients are charged into a 4 ml vial and closed with headspace volume as constant as possible to allow for comparison and the sample is kept at room temperature and tilted at regular time intervals to determine whether the material still flows. If no flow is observed during tiling, the vial is held upside down and if no further flow occurs the materials is gelled.

Gloss was measured using a handheld Micro-Tri-Gloss meter from BYK Instruments. Measurements were taken at 60 degrees in three different locations on the film and the average is reported.

Pencil Hardness was performed according to the ISO 15184 test method. The pencil hardness rating scale is as follows: [Soft] 9B-8B-7B-6B-5B-4B-3B-2B-B-HB-F-H-2H-3H-4H-5H-6H-7H-8H-9H [Hard].

Acetone removal was determined by placing a cotton ball in the center of the nail polish coating. Acetone was added to the cotton ball until the liquid layer barely showed at the edge of the cotton ball. After starting the test, the cotton ball was briefly lifted to examine the coating integrity below the cotton ball. In order to adjust for the acetone evaporation throughout the test time period, additional acetone was added to the cotton ball to maintain that light liquid layer at the edge of the cotton ball. The time at which the surface integrity became disrupted was determined as the test end point.

### Inventive Example 1

The TBMA DMC solution in ethanol prepared under example 1 was tested as dormant carbamate initiator. In a vial, 1.0 g of the malonate resin prepared under example 1 was mixed with 1.5 g of di-trimethylolpropane tetraacrylate (DTMPTA) and then 1.148 g of the TBMA DMC initiator solution in ethanol was added. The complete formulation was mixed well and then a test film was applied on a glass substrate to test curing behavior. The coating film become tack-free within 5 minutes and the gel time of the material in the vial was longer than 24 hours. The carbamate is a dormant initiator.

### Inventive Example 2

A mixture was prepared in a vial combining 1.0 g of the malonate resin prepared under example 5 and 1.27 g of trimethylolpropane triacrylate (TMPTA). Next, 1.3 g of the TBMA DMC carbamate solution prepared of example 2 was added to the vial and the liquid was mixed well. A film was then applied onto a glass slide and the coating became tack free within 5 minutes. No gelation of the material in the vial was observed after three weeks of aging. Another film was prepared of this aged mixture and again the coating cured within 5 minutes. Hence, the carbamate is an effective dormant initiator.

### Inventive Example 3

Three TBA DMC solutions in anhydrous ethanol were compared and tested as dormant initiator. Initiator I was a TBA DMC solution in anhydrous ethanol prepared per the cation exchange procedure as set forth in example 1. Initiator II was prepared in example 3 from TBA OH and DMA DMC in a 1.0:1.1 molar ratio, and initiator III was prepared using a 1.0:1.5 molar ratio. A resin mixture was formulated from the malonate resin prepared under example 5 and TMPTA. The molar ratio for malonate methylene CH2 to TMPTA to initiator was 3:2:0.2 respectively. The percent water for the carbamate initiator obtained by means of neutralization was calculated from reaction stoichiometry and is based as percentage of the total crosslinkable formulation. Anhydrous ethanol was added as necessary to arrive at a comparable percent solvent content. The ethanol solvent content is also based on total weight of the crosslinkable formulation. The tack free time of a film applied on a glass substrate was assessed as well as gel time. Data provided in table 1 show that all three carbamate solutions are active as a dormant carbamate initiator and they become effective once the initiator activates by means of film formation.

TABLE 1

| Carbarmate | Wt. % | Wt. % | Tack free | Gel |
|---|---|---|---|---|
| initiator | Water | Ethanol | time | time |
| I | 0.0 | 32.7 | 140 sec | >72 h |
| II | 0.2 | 31.8 | 140 sec | >72 h |
| III | 0.2 | 31.2 | 120 sec | >72 h |

### Inventive Example 4

Initiator II was prepared in example 3 from TBA OH in ethanol, 1-propanol or 2-propanol with TBA OH to DMA DMC in a 1.0:1.1 molar ratio. Initiator III was prepared also in ethanol, 1-propanol or 2-propanol and the TBA OH to DMA DMC molar ratio employed was 1.0:1.5 respectively. The initiators were used either without addition of additional water, or water was added to these initiator solutions to target about 1.2 wt % water content based as percentage of the final crosslinkable formulation. At 1.2 wt % water content, there is about 4.5 moles of water per mole of initiator present. Similarly, a 10-15 wt % alcohol content was targeted based on the final crosslinkable formulation. A resin mixture was formulated from the malonate resin prepared under example 5 and TMPTA. The molar ratio for malonate methylene CH2 to TMPTA to initiator was chosen at 3:2:0.2 respectively. Films were applied on a glass substrate to test for tack free time. Results shown in table 2 indicate that both carbamate initiators are dormant while the formulation remains in the vial, while good activation occurs once a film is applied. The coating formulation in ethanol shows a longer gel time than 1-propanol and 2-propanol for initiator II, but this can be improved by adding a little additional water and solvent. Addition of additional DMA DMC to the carbamate initiator system also improves gel time when initiator II and III are compared but this does not seem to significantly impact tack free time.

TABLE 2

| Carbamate | | Wt. % | Wt. % | Tack free | | Gel | |
|---|---|---|---|---|---|---|---|
| initiator | Solvent | Water | Solvent | time | | time | |
| II | Ethanol | 0.3 | 8.3 | 90 | sec | >16 | h |
| II | 1-propanol | 0.3 | 8.3 | 90 | sec | 6-8 | h |
| II | 2-propanol | 0.3 | 8.3 | <90 | sec | 6-8 | h |
| II | Ethanol | 1.2 | 12.9 | 140 | | >16 | h |
| II | 1-propanol | 1.2 | 12.9 | 140 | | >16 | h |
| II | 2-propanol | 1.2 | 12.9 | 140 | | >16 | h |
| III | Ethanol | 0.3 | 9.1 | 90 | sec | >24 | h |
| III | 1-propanol | 0.3 | 9.1 | 90 | sec | >24 | h |
| III | 2-propanol | 0.3 | 9.1 | <90 | sec | >24 | h |

| | | | | | | |
|---|---|---|---|---|---|---|
| III | Ethanol | 1.2 | 12.9 | 120 | >24 | h |
| III | 1-propanol | 1.2 | 12.9 | 120 | >24 | h |
| III | 2-propanol | 1.2 | 12.9 | 130 | >24 | h |

### Comparative Example 1 (Versus Inventive Example 3 and 4)

Diethylcarbonate derived catalysts were prepared in ethanol, 1-propanol and 2-propanol as per example 4. Water content was fixed at either 0 wt. %, or water was added to the catalyst solutions to target about 1.2 wt. % water content based as percentage of the final crosslinkable formulation. At 1.2 wt. % water content, there is about 4.5 moles of water per mole of blocked base catalyst present. The catalyst solutions were tested as blocked catalyst in a resin mixture formulated from the malonate resin prepared under example 5 and TMPTA using a molar ratio for malonate methylene CH2 to TMPTA to catalyst of 3:2:0.2 respectively, which is similar to inventive examples 3 and 4. Results shown in table 3 indicate that the carbonate solutions are not active as a blocked catalyst in ethanol, 1-propanol or 2-propanol in the absence of water, and even addition of water up to 1 wt. % of the total formulation does not lead to effective blocking of the carbonate base catalyst in these solvents. No tack free time could be measured because the resin—carbonate catalyst mixture polymerized immediately and an instant gel was formed.

TABLE 3

| Carbonate | | | | Tack free | Gel |
|---|---|---|---|---|---|
| catalyst | Solvent | % Water | % Solvent | time | time |
| DEtC | Ethanol | 0.0 | 14.4 | Instant gel | <30 sec |
| DEtC | 1-propanol | 0.0 | 14.4 | Instant gel | <30 sec |
| DEtC | 2-propanol | 0.0 | 14.4 | Instant gel | <30 sec |
| DEtC | Ethanol | 1.2 | 14.3 | Instant gel | <30 sec |
| DEtC | 1-propanol | 1.2 | 14.3 | Instant gel | <30 sec |
| DEtC | 2-propanol | 1.2 | 14.3 | Instant gel | <30 sec |

### Comparative Example 2 (Versus Inventive Example 3 and 4)

The experiment of comparative example 1 was repeated except that dimethylcarbonate catalyst solutions were used as prepared per example 4. Results presented in table 4 show that the blocking is not effective in these solvents when water is absent, and even addition of water up to about 1 wt. % of the total formulation does not produce an effective blocking effect.

TABLE 4

| Carbonate | | Wt. % | Wt. % | Tack free | Gel |
|---|---|---|---|---|---|
| catalyst | Solvent | Water | Solvent | time | time |
| DMeC | Ethanol | 0.0 | 12.9 | Instant gel | <30 sec |
| DMeC | 1-propanol | 0.0 | 12.9 | Instant gel | <30 sec |
| DMeC | 2-propanol | 0.0 | 12.9 | Instant gel | <30 sec |
| DMeC | Ethanol | 1.2 | 12.9 | Instant gel | <30 sec |
| DMeC | 1-propanol | 1.2 | 12.9 | Instant gel | <30 sec |
| DMeC | 2-propanol | 1.2 | 12.9 | Instant gel | 45 sec |

### Inventive Example 5

The experiment of inventive example 4 is repeated except methanol is used as solvent for initiator II and III and results are shown in table 5. Both carbamate solutions are effective and carbamate is active as a dormant initiator that activates once the coating formulations is applied as a film.

TABLE 5

| Carbamate | | Wt. % | Wt. % | Tack free | Gel |
|---|---|---|---|---|---|
| initiator | Solvent | Water | Methanol | time | time |
| II | Methanol | 0.3 | 8.3 | <90 sec | 4 days |
| III | Methanol | 0.3 | 9.1 | <90 sec | >6 days |

### Comparative Example 3 (Versus Inventive Example 5)

A similar experiment is carried out as comparative examples 1 and 2 for DEtC and DMeC respectively, except methanol is used as the solvent and results are shown in table 6.

TABLE 6

| Carbonate | | | | Tack free | Gel | |
|---|---|---|---|---|---|---|
| catalyst | Solvent | % Water | % Methanol | time | time | |
| DEtC | Methanol | 0.0 | 14.3 | <90 sec | 16 | h |
| DEtC | Methanol | 1.2 | 14.3 | <90 sec | 6 | days |
| DMeC | Methanol | 0.0 | 13.0 | <90 sec | 16 | h |
| DMeC | Methanol | 1.2 | 12.9 | <120 sec | >6 | days |

### Inventive Example 6

About 1 ml of the initiators prepared in example 1 and example 3 (1:1.1 ratio of TBMA OH to DMA DMC) with as-is concentration is each added to a 2 ml clear vial. DMA DMC is also added to a vial for comparison. The carbamate solutions obtained via ion exchange are essentially free of water, while the carbamate solutions obtained via neutralization as per example 3 contain an equal molar amount of water per amount of initiator. Next, 2 drops of phenolphthalein indicator is added to the solution and mixed well. After mixing, the color is observed and a pink color means the solutions is basics, while a colorless solution means no base if present. As expected, the TBMA OH solution has a pink color and is basic, but the carbamate solutions are all colorless. Hence, the dormant carbamate initiator solutions are not basic.

TABLE 7

| | | Solution | |
|---|---|---|---|
| Materials | Solvent | color | Comment |
| DMA DMA | — | Colorless | — |
| TBMA OH | Methanol | Pink | Active base |
| TBMA OH + DMA DMC | Methanol | Colorless | Dormant initiator |
| TBMA OH + DMA DMC | Ethanol | Colorless | Dormant initiator |
| TBMA OH + DMA DMC | 1-propanol | Colorless | Dormant initiator |
| TBMA OH + DMA DMC | 2-propanol | Colorless | Dormant initiator |
| Ion exchanged TBMA DMC | Ethanol | Colorless | Dormant initiator |
| Ion exchanged TBMA DMC | 1-propanol | Colorless | Dormant initiator |
| Ion exchanged TBMA DMC | 2-propanol | Colorless | Dormant initiator |

### Comparative Example 4 (Versus Inventive Example 6)

About 1 ml of the catalysts prepared in example 4 using TBMA OH and DEtC with as-is concentration is each added to a 2 ml clear vial. Next, 2 drops of phenolphthalein indicator is added to the solution and mixed well. After mixing the final color change is observed as either pink or colorless and results are tabulated in table 8. A pink colored solution means the solution is basic and a colorless solutions means that the base is blocked from activity. Only the base in methanol is blocked by the carbonate but the base was not blocked by the carbonate in the other alcohols and remained active as base.

TABLE 8

| | | Solution | |
|---|---|---|---|
| Materials | Solvent | color | Comment |
| TBMA OH | Methanol | Pink | Active base |
| TBMA OH + DEtC | Methanol | Colorless | Blocked catalyst |
| TBMA OH + DEtC | Ethanol | Pink | Active base |
| TBMA OH + DEtC | 1-propanol | Pink | Active base |
| TBMA OH + DEtC | 2-propanol | Pink | Active base |

### Inventive Example 7

The dormant carbamate initiator was employed in a cross-linkable coating composition as to formulate a nail polish system. The system utilized three coatings; a basecoat/primer, a color coat, and a topcoat to allow for comparison against commercial UV nail gel and conventional (solvent borne) nail polish systems, which also employ a three coat approach. Two nail polish systems (inventive example 7.1 and 7.2) were formulated based on the inventive crosslinkable composition.

### Carbamate Initiator Synthesis:

Most of the methanol solvent from a 40 g tetrabutylammonium hydroxide (TBA OH) solution in methanol (1 M) is removed with a rotary evaporator in about 30 minutes at room temperature. Next, 40 grams of ethanol is added and most of the solvent is again removed in a similar manner. This procedure is repeated at least two more times until the methanol effectively has been replaced. The complete removal of methanol is confirmed by H NMR analysis. Next, 25 g of the TBAOH in EtOH (1.34 mmol base/g solution) solution is mixed with 6.4 g DMA DMC at room temperature and stirred for 1 hour using magnetic stirrer. The final light yellow solution has an initiator concentration of 1.38 mmol/g sample.

Base Coat Formulations:

two different base coats were formulated.

Base Coat a;

formula ingredients: 4.55 wt. % of malonate resin (I) of example 5; 40.91 wt. % of malonate resin (II) of example 6; 19.91 wt. % of DTMPTA; 9.10 wt. % of butyl acetate (BA); 9.10% of ethyl acetate (EA); 1.83 wt. % of an alkyl ethoxylate wetting agent; and 14.60 wt. % of carbamate initiator. All the ingredients except the initiator were weighed into a 20 ml vial. The vial was capped and the mixture shaken until visually homogenous. The dormant carbamate initiator was then weighed into the mixture. The final mixture was capped and shaken for 30 seconds, and then applied using a 3 mil Bird type film applicator on a vitronail panel substrate.

Base Coat B;

formula ingredients: 7.28 wt. % of malonate resin (III) of example 7; 40.95 wt. % of malonate resin (II) of example 6; 19.93 wt. % of DTMPTA; 6.37 wt. % of BA; 9.10% of EA; 1.82 wt. % of an alkyl ethoxylate wetting agent; and 14.56 wt. % of carbamate initiator. All the ingredients except the initiator were weighed into a 20 ml vial. The vial was capped and the mixture shaken until visually homogenous. The dormant carbamate initiator was then weighed into the mixture. The final mixture was capped and shaken for 30 seconds, and then applied using a 3 mil Bird type film applicator on a vitronail panel substrate.

Color Coat Formulation:

only one color coat A was formulated.

A Colorant Pigment Dispersion was prepared first. Formula ingredients: 62.65 wt. % of malonate resin (I) of example 5; 37.35 wt. % of Chemours TS-6200 white pigment. The resin was added to the stainless steel mixing vessel. Mixing of the resin was begun using a high speed dispersion mixer at 1.5 mm/s using a 50 mm mixing blade. The TS-6200 pigment was poured at a medium rate into the mixing resin. After all of the TS-6200 had been added, the mixing speed was increased to 7.85 m/s and held constant for 10 min. At the end of mixing, the mixture was poured into a storage jar and sealed.

Color coat A was formulated as follows: formula ingredients: 25.00 wt. % of the Colorant Pigment Dispersion; 9.15 wt. % of malonate resin (IV) of example 8; 6.10 wt. % malonate resin (II) of example 6; 35.37 wt. % of DTMPTA; 12.20 wt. % of BA; 2.43 wt. % of an alkyl ethoxylate wetting agent; and 9.75 wt. % of carbamate initiator. All the ingredients except the initiator were weighed into a 20 ml vial. The vial was capped and the mixture shaken until visually homogenous. The dormant carbamate initiator was then weighed into the mixture. The final mixture was capped and shaken for 30 seconds, and then applied over the dried base coat using a 3 mil Bird type film applicator.

Top Coat Formulations:

two different top coats were formulated.

Top Coat a;

formula ingredients: 18.12 wt. % of malonate resin (I) of example 5; 10.87 wt. % of malonate resin (IV) of example 8; 7.25 wt. % of of malonate resin (II) of example 6; 42.03 wt. % of DTMPTA; 7.25 wt. % of BA; 1.45 wt. % of 1,3-butanediol (BD); 1.44 wt. % of an alkyl ethoxylate wetting agent; and 11.59 wt. % of carbamate initiator. All the ingredients except the initiator were weighed into a 20 ml vial. The vial was capped and the mixture shaken until visually homogenous. The dormant carbamate initiator was then weighed into the mixture. The final mixture was capped and shaken for 30 seconds, and then applied over the dried color coat using a 3 mil Bird type film applicator.

Top Coat B;

formula ingredients: 28.82 wt. % of malonate resin (III) of example 7; 10.37 wt. % of malonate resin (IV) of example 8; 6.91 wt. % of malonate resin (II) of example 6; 40.08 wt. % of DTMPTA; 1.38 wt. % of BD; 1.38 wt. % of an alkyl ethoxylate wetting agent; and 11.06 wt. % of carbamate initiator. All the ingredients except the initiator were weighed into a 20 ml vial. The vial was capped and the mixture shaken until visually homogenous. The dormant carbamate initiator was then weighed into the mixture. The final mixture was capped and shaken for 30 seconds, and then applied over the dried color coat using a 3 mil Bird type film applicator. Commercial systems: the commercial systems were applied in a similar manner also on vitronail substrate panels and cured as per instructions and procedures common to the industry.

The various coats of the nail coating systems are summarized in Table 9.

TABLE 9

| Nail polish system | Base coat | Color coat | Top coat |
|---|---|---|---|
| Inventive 7.1 | Base coat A | Color coat A | Top coat A |
| Inventive 7.2 | Base coat B | Color coat A | Top coat B |
| UV nail gel | OPI GelColor | OPI GelColor | OPI GelColor |
| | Base coat | Pink Flamenco | Top coat |
| | | Color coat | |
| Conventional | Revlon Color- | Nina Ultra Pro | Revlon Color- |
| nail polish | Stay Gel- | Mariachi | stay Gel Envy |
| | Smooth | Color coat | Diamond |
| | Base coat | | Top Coat |

Nail polish performance test results are shown in the table 10. Inventive coatings 7.1 and 7.2 exhibit comparable gloss and tack free dry times compared to the commercial references. The pencil hardness of these coatings are substantially greater than either of the references used in this testing. The acetone removal times of both inventive coatings were significantly faster than the commercial UV nail gel coating system. The conventional nail polish system was easiest to remove as expected, but the film was also extremely soft.

TABLE 10

| | | | | Performance whole system | | |
|---|---|---|---|---|---|---|
| | Tack free time individual coat | | | | | Acetone |
| | Base coat | Color coat | Top coat | | Pencil | removal time |
| Nail polish system | (min) | (min) | (min) | 60° gloss | hardness | (min) |
| Inventive 6.1 | 3 | 3.5 | 5.5 | 75 | 6.5H | 13 |
| Inventive 6.2 | 2.3 | 3.8 | 5.3 | 72 | 8H | 20 |
| UV nail gel | 4 | 3.5 | 3.5 | 73 | 3.5H | 27 |
| Conventional nail | 1.25 | 2.5 | 1.3 | 81 | 9B | 0.5 |
| polish | | | | | | |

### Inventive Example 8

The dormant carbamate initiator is used to cure a mixture of the acetoacetate modified polyol of example 9 and DTMPTA. A vial is charged with 46 wt. % acetoacetate modified polyol, 0.74 wt. % alkyl ethoxylate wetting agent, 36.86 wt. % DTMPTA and 9.2 wt. % BA. The vial was stirred until homogenous. Next, a carbamate initiator type II was prepared as in example 3 (46% in ethanol) and 7.4 wt. % of this initiator was then weighed into the coating mixture. The final mixture was capped and shaken for 30 seconds, and applied on a polycarbonate sheet using a 3 mil Bird type film applicator. The resulting coating cured quickly and was tack free in 20 minutes and had a glossy appearance (94 at 60°) and the gel time was 65 minutes.

As control, 45.87 wt. % of the STEPANPOL® PC-2011-225 polyol resin, 0.69 wt. % EFKA SL-3288; and 18.35 wt. % BA were weighed into a 20 ml vial and mixed. Next, 34.40% Basonat HB 100 isocyanate curative was added and the mixture stirred again before 0.69 wt. % Borchi-Kat 24 urethane catalyst was added and stirred in. A film was drawn down using a 3 mil Bird bar type film applicator. The resulting glossy coating (93 at 60°) cured tack free in 50 minutes but the gel time was only 2 minutes.

### Inventive Example 9

Dormant carbamate initiator type II was prepared in example 3 from TBA OH in ethanol and varying amounts of this initiator system was used to assess cure speed using the malonate resin prepared under example 5 and TMPTA. The molar ratio for malonate methylene CH₂ to TMPTA was fixed at 3:2, while the ethanol content was kept as constant as possible at about 10 wt % of the final formulation. The amount of initiator used is expressed as mole percent relative to the number of protons that can be abstracted to form activated Michael donor species. Films were applied on glass substrates to test tack free time and these are summarized in table 11. Some of the films with higher initiator concentrations gave a wrinkled appearance as the solvent content/package was not optimal in view of such fast cure speeds, however, increased carbamate initiator content provided faster cure rates.

TABLE 11

| | | | | | | |
|---|---|---|---|---|---|---|
| Carbamate initiator (mole %) | 0.83 | 1.67 | 3.33 | 6.67 | 10 | 13.33 |
| Tack free time (sec) | 600 | 455 | 328 | 213 | 154 | 100 |

### Inventive Example 10

A diethylene glycol monomethyl ether (DEGMEE) end capped condensation copolymer of diethyl malonate and 1,3-propanediol was prepared and this resin had a number average molecular weight of about 1,200 and a PDI of 1.3 (calculated based a Polystyrene GPC calibration). This resin is abbreviated as DEM-PD-DEGMEE and was used to formulate three different clear coat systems that contained different quantities of 1,2,4-triazole.

In a specific clear coat example, 0.97 g of the DEM-PD-DEGMEE resin was combined with 1.24 g of DTMPTA in a 20 ml glass vial. The mixture was stirred by hand using a spatula to make it homogenous. After this, 0.29 g of BA, and 0.08 g of a 30% 1,2,4-triazole in EtOH solution was added. The vial was sealed and then vigorously shaken until homogenous (1-3 min.). Test panels to be coated were placed into position at this point. Bird Bars (3 mil) for the coating application were made ready. The glass vial was unsealed and 0.35 g of dormant carbamate initiator of example 10 is added. The lid was placed back on the vial. The complete mixture was vigorously shaken to make it homogenous (1-3 min.).

Table 12 shows formulations for the three different topcoats that contain different quantities of 1,2,4-triazole. The mole amount of triazole is expressed as percent relative to the mole amount of initiator.

TABLE 12

| Formula Ingredients | Topcoat A | Topcoat B | Topcoat C |
|---|---|---|---|
| DEM-PD-DEGMEE resin | 1.00 | 0.97 | 1.08 |
| DTMPTA | 1.28 | 1.24 | 1.29 |
| BA | 0.30 | 0.29 | 0.33 |
| 1,2,4-Triazole | 0.00 | 0.08 | 0.16 |
| (30.0% in EtOH) | | | |
| Carbamate initiator | 0.35 | 0.35 | 0.38 |
| (27.7% in EtOH) | | | |
| % Triazole content vs | 0% | 101% | 190% |
| carbamate catalyst | | | |

The three topcoat formulations were first evaluated by themselves to assess the effects of adding incremental amounts of 1,2,4-triazole. Films were drawn down at 3 mils wet film thickness on 4″×6″ polycarbonate panels using a 3 mil Bird bar. Results presented in table 13 show that compared to Topcoat A with no added Triazole, Topcoat B has a similar tack time, while Topcoat C has a significantly longer tack free time (−148% when compared to Control A). Above a certain threshold amount, added 1,2,4-triazole delays cure speed and increases tack free time and open time.

TABLE 13

| | | | | | % Im- |
|---|---|---|---|---|---|
| Conventional | | Top | 1,2,4- | Tack | provement |
| Color Coat | | Coat | Triazole | Time | in Tack |
| Nail Enamel | Supplier | Layer | Amount | (min) | Time |
| none | — | Topcoat A | 0.0% | 2.7 | — |
| none | — | Topcoat B | 24.7% | 2.7 | 0% |
| none | — | Topcoat C | 45.7% | 6.7 | −148% |
| Insta-Dry 291 | Sally Hansen | Topcoat A | 0.0% | 30.0 | — |
| ASAP Apple | | | | | |
| (RED) | | | | | |
| Insta-Dry 291 | Sally Hansen | Topcoat C | 45.7% | 8.0 | +73% |
| ASAP Apple | | | | | |
| (RED) | | | | | |
| Make it Work | Defy & Inspire/ | Topcoat A | 0.0% | 8.5 | — |
| 280 (Green) | Beauty Partners | | | | |
| | LLC | | | | |
| Make it Work | Defy & Inspire/ | Topcoat B | 24.7% | 3.0 | +65% |
| 280 (Green) | Beauty Partners | | | | |
| | LLC | | | | |

The three topcoats were then evaluated by drawing them down over panels that were already primed with conventional color coat nail polish. Conventional nail polish was first applied to polycarbonate test panels by drawing them down at a 3 mil wet film thickness. These were allowed to dry under ambient laboratory conditions for about 10 minutes prior to coating them with the topcoat formulas of table 12. The topcoat formulas were applied at 3 mils wet film thickness on top of the dried conventional color coatings. Results of the tack free time evaluations of the topcoats applied over the conventional color coats are shown in table 13. Addition of 1,2,4-triazole greatly sped up cure characteristics when inventive clear coat is applied over conventional colored nail polish.

### LIST OF CHEMICAL ACRONYMS USED IN THE EXAMPLES

BA butyl acetate
BD 1,3-butanediol
EA ethyl acetate
DEM diethyl malonate
DEtC diethyl carbonate
DMA DMC dimethylammonium dimethylcarbamate
DMeC dimethylcarbonate
DTMPTA di-trimethylolpropane tetraacrylate
Gly glycerol
PEG 300 polyethylene glycol, Mw=300
PtB potassium t-butoxide
tBAA tert-butyl acetoacetate
TBA DMC tetrabutylammonium dimethylcarbamate
TBMA tributylmethylammonium
TBMA Cl tributylmethylammonium chloride
TBMA DMC tributylmethylammonium dimethylcarbamate
THF tetrahydrofuran
TMP trimethylolpropane
TMPTA trimethylolpropane triacrylate

The present disclosure may be embodied in other specific forms without departing from the spirit or essential attributes of the invention. Accordingly, reference should be made to the appended claims, rather than the foregoing specification, as indicating the scope of the disclosure. Although the foregoing description is directed to the preferred embodiments of the disclosure, it is noted that other variations and modification will be apparent to those skilled in the art, and may be made without departing from the spirit or scope of the disclosure.

1. A clear coating composition comprising:
(a) a crosslinkable coating composition comprising:
ingredient A that has at least two protons that can be activated to form a Michael carbanion donor;
ingredient B that functions as a Michael acceptor having at least two ethylenically unsaturated functionalities each activated by an electron-withdrawing group; and
a dormant carbamate initiator of Formula (1) 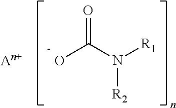
wherein R₁ and R₂ can be independently selected from hydrogen, a linear or branched substituted or unsubstituted alkyl group having 1 to 22 carbon atoms; 1 to 8 carbon atoms; 1 to 3 carbon atoms; and Aⁿ⁺ is a cationic species or polymer and n is an integer equal or greater than 1 with the proviso that Aⁿ⁺ is not an acidic hydrogen;
an ingredient D having one or more reactive protons that are more acidic than the two protons of ingredient A, with respect to pKa;
at least one organic solvent;
and optionally further comprising ammonium carbamate (H₂NR₁R₂⁺⁻OC═ONR₁R₂); and
with the proviso that the clear coating composition is free of FD&C or D&C dyes, lakes or pigments.

2. The clear coating composition according to claim 1, wherein the ingredient A is independently selected from a malonate group containing compound, a malonate group containing oligomer, a malonate group containing polymer, an acetoacetate group containing compound, an acetoacetate group containing oligomer, an acetoacetate group containing polymer or combinations thereof.

3. The clear coating composition according to claim 2, wherein the malonate group containing compound, malonate group containing oligomer, malonate group containing polymer, an acetoacetate group containing compound, acetoacetate group containing oligomer, or acetoacetate group containing polymer are each selected from the group consisting of: polyurethanes, polyesters, polyacrylates, epoxy polymers, polyamides, polyesteramides or polyvinyl polymers, wherein such compounds, oligomers or polymers have a malonate group or acetoacetate group located in a main chain of such compound or oligomer or polymer or a side chain of such compound or oligomer or polymer.

4. The clear coating composition according to claim 3, wherein ingredient B is selected from the group consisting of acrylates, fumarates, maleates and combinations thereof.

5. The clear coating composition according to claim 4, wherein the acrylate is independently selected from the group consisting of hexanediol diacrylate, trimethylol propane triacrylate, pentaerythritol triacrylate, di-trimethylolpropane tetraacrylate, bis(2-hydroxyethyl acrylate), trimethylhexyl dicarbamate, bis(2-hydroxyethyl acrylate) 1,3,3-trimethylcyclohexyl dicarbamate, bis(2-hydroxylethyl acrylate) methylene dicyclohexyl dicarbamate and combinations thereof.

6. The clear coating composition according to claim 4, wherein ingredient B is independently selected from the group consisting of polyesters, polyurethanes, polyethers and/or alkyd resins each containing at least two pendant ethylenically unsaturated groups each activated by an electron-withdrawing group.

7. The clear coating composition according to claim 4, wherein ingredient B is independently selected from the group consisting of polyesters, polyurethanes, polyethers and/or alkyd resins each containing at least one pendant acryloyl functional group.

8. The crosslinkable coating composition according to claim 1, wherein the one or more reactive protons of ingredient D are less acidic than the ammonium cation of the optional ammonium carbamate, with respect to pKa.

9. The clear coating composition according to claim 1, further comprising water at a weigh percent selected from the group consisting of less than 10 wt. % water; less than 5 wt. % water; less than 1 wt. % water; less than 0.1 wt. % water; less than 0.01 wt. % water.

10. The clear coating composition according to claim 1, wherein the organic solvent is independently selected from the group consisting of an alcohol, ester, ether, glycol ether, ketone, aromatic and combinations thereof.

11. The clear coating composition according to claim 10, wherein the solvent is independently selected from the group consisting of acetone, ethyl acetate, butyl acetate, isopropyl alcohol, ethanol, methyl ethyl ketone, toluene, hexane, and mixtures thereof.

12. The clear coating composition according to claim 1, wherein A⁺ⁿ is a monovalent quaternary ammonium compound of Formula (2) 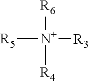
wherein R₃, R₄ and R₅ are independently selected from linear or branched alkyl chains having from 1 to 22 carbon atoms; or 1 to 8 carbon atoms or 1 to 6 carbon atoms and combinations thereof; and wherein R₆ is independently selected from the group consisting of: methyl, an alkyl group having from 2 to 6 carbon atoms or a benzyl group.

13. The clear coating composition according claim 12, wherein the dormant carbamate initiator initiates Michael Addition to achieve crossing linking when the clear coating composition is applied to a surface.

14. The clear coating composition according to claim 13, wherein ingredient A, ingredient B, ingredient D, the organic solvent and the dormant carbamate initiator are contained in a container having two or more chambers, which are separated from one another.

15. The clear coating composition according to claim 14, wherein ingredient A and ingredient B are contained in separate chambers to inhibit any reaction.

16. The clear coating composition according to claim 14, wherein the dormant carbamate initiator is contained in the chamber having ingredient A, and optionally containing ingredient D, CO₂ and/or ammonium carbamate.

17. The clear coating composition according to claim 14, wherein ingredient A, ingredient B and optionally ingredient D are contained in the same chamber and the dormant carbamate initiator is contained in a separate chamber to inhibit any reaction and said separate chamber optionally containing CO₂ and/or ammonium carbamate.

18. The clear coating composition according to claim 14, wherein ingredient A and ingredient B and dormant carbamate initiator are contained in a container having a single chamber, wherein the container optionally contains ingredient D, CO₂ and/or ammonium carbamate.

19. The clear coating composition according to claim 12, further comprising a rheological additive to modify rheology.

20. The clear coating composition according to claim 19, further comprising a wetting agent.

21. The clear coating composition according to claim 20, further comprising an adhesion promotor.

22-43. (canceled)
